(19) United States
(12) Patent Application Publication
(10) Pub. No.: US 2004/0001791 A1
(43) Pub. Date: Jan. 1, 2004

(54) Composition, method of use, and devices for the treatment of onychomycosis

(76) Inventor: Kenneth T. Zeiler, Irving, TX (US)

Correspondence Address: GREENLEE WINNER AND SULLIVAN P C, 5370 MANHATTAN CIRCLE, SUITE 201, BOULDER, CO, 80303, US

(21) Appl. No.: 10/440,468

(22) Filed: May 16, 2003

Related U.S. Application Data
(63) Continuation-in-part of application No. PCT/US01/29438, filed on Sep. 19, 2001.
(60) Provisional application No. 60/249,381, filed on Nov. 16, 2000.
(60) Provisional application No. 60/286,781, filed on Apr. 26, 2001.
(60) Provisional application No. 60/421,257, filed on Oct. 25, 2002.

(51) Int. Cl. A61K 33/34; A61K 7/04

(52) U.S. Cl. 424/61; 424/637

(57) ABSTRACT

The invention is a method for treating onychomycosis in humans comprising contacting a fungal infected nail with a composition comprising an effective amount of a copper salt. Treating human nails with the copper salt composition can be used to detect fungal infection before physical symptoms are presented. Fungal infection is also prevented by pre-treatment of nails with the copper salt composition.

### CROSS-REFERENCE TO RELATED APPLICATIONS

This application is a continuation-in-part of PCT Application No. PCT/US01/29438 filed Sep. 19, 2001, which claims priority to U.S. Provisional Application No. 60/249,381 filed Nov. 16, 2000 and U.S. Provisional Application No. 60/286,781 filed Apr. 26, 2001. This application also claims priority to U.S. Provisional Application No. 60/421,257 filed Oct. 25, 2002. All of these applications are incorporated by reference to the extent not inconsistent with the disclosure hereof.

### BACKGROUND OF THE INVENTION

This invention relates to a composition for treating fungal-infected human nails.

Nail fungal infections (also known as onychomycosis, tinea unguium, or ringworm of the nail; hereinafter referred to as “onychomycosis”) are caused by a fungal invasion of the keratinous structure of a fingernail or toe nail by a variety of fungi including Trychophyton sp., Microsporum sp., Epidermaphyton sp.; Candida sp., Trichosporon sp., Geotrichum sp., Scopulariopsis sp., Aspergillus sp., Acremonium sp., Fusarium sp., Phyllosticta sp., Hendersonula sp., and Cheatomium sp. A fungal infected nail typically has a yellowish color that starts at one comer of the nail and spreads towards the cuticle. Over time, the infected nail presents as a thickened, lusterless, discolored nail, often with keratotic debris accumulated under the tip. Left untreated, the infected nail plate often becomes brittle, flaking, crumbling, friable, striated, deformed, separated, or completely destroyed. While onychomycosis is generally not painful, the unsightly appearance of the infected nail can be a source of embarrassment for most patients. For diabetics or immunosuppressed patients, an infected nail left untreated can result in severe infection.

Various systemic and topical preparations have been used to treat onychomycosis. Generally, the course of treatment is long-term and accompanied by a high relapse rate. Itraconazole, which inhibits the cytochrome P-450-dependent synthesis of erosterol, a vital component of fungal cell membranes, is available for oral administration. The recommended course of treatment with itraconazole for an infected toe nail is twelve weeks. For an infected fingernail, two one-week pulses of orally administered itraconazole separated by three weeks are recommended. Griseofulvin is an oral preparation that binds newly formed keratin, thus making new nail tissue resistant to fungal infection. Recommended treatment involves trimming away infected nail tissue as the nail grows out and requires at least four months of treatment for an infected fingernail and at least six months for an infected toe nail. Because griseofulvin is derived from Penicillium, the possibility of cross-sensitivity with penicillin exists, limiting its use to individuals not allergic to penicillins. Terbinafine hydrochloride is an oral preparation that prevents fungal growth by inhibiting squalene epoxidase and blocking fungal cell membrane synthesis. Recommended treatment of terbinafine hydrochloride is six weeks for an infected fingernail and 12 weeks for an infected toe nail. Topical antifungal agents include imidazoles or triazoles such as clotrimazole and ketoconazole, ciclopirox, amphotericin-B, gentian violet, resorcinol, and iodine. Despite the current availability of antifungal agents for treating onychomycosis, new compositions that are less expensive to administer with a minimal relapse rate are desired.

The antifungal use of various copper salts is well known in the area of agriculture and aquaculture. For example, copper sulfate, copper carbonate, copper oxychloride, copper ammonium complex, copper oxide, copper hydroxide, copper naphthenates, and copper quinolinates have all been reported as effective fungicidal agents. The antifungal properties of copper sulfate solutions have been known for over 230 years and were utilized to eliminate wheat bunt (Tilletia sp.) and potato blight, the cause of the Irish Potato Famine. The oldest of the treatments to prevent the growth of Phytophthora infestans, the causative agent in potato blight, are known as the Bordeaux mixture and the Burgundy mixture. Both solutions are prepared for spraying on potato plants to prevent blight according to a rigorous schedule synchronized to the fungus lifecycle. A 2% Bordeaux mixture is most commonly used for potatoes and consists of a 2:1 mixture of copper sulfate and hydrated lime in 100 gallons of water. A 1% Burgundy mixture consists of a 4:5 mixture of copper sulfate and sodium bicarbonate in 40 gallons of water. Despite the availability of synthetic antifungal agents to prevent potato blight, organic farmers still utilize the Bordeaux mixture and the Burgundy mixture because they are safe, inexpensive, and easy to use.

In U.S. Pat. No. 4,822,595 issued Apr. 18, 1989, Corliss, et al. disclose a hoof lotion that contains linseed oil as a dispersing agent, lanolin as a moisturizer, turpentine as a drying agent, iodine as an antiseptic agent, pine tar as a sticking agent, hydrogen peroxide as an antibacterial agent and copper sulfate as a fungicidal agent. Copper sulfate is described as being 0.0053% by weight of the hoof lotion. Corliss, et al. described the utility of the claimed hoof lotion as killing and preventing fungal growths and healing cracks, brittle surfaces and hoof dryness.

Undecylenic acid as well as its copper, calcium and zinc salts and combinations thereof have been reported as antifungal agents for use in the treatment of athlete's foot (tinea pedis). However, the foot creams, sprays, and ointments containing undecylenates have been limited to the treatment of skin fungal infections and specifically disclaimed as treatment for fungal nail infections.

There is a need in the art for an effective easy-to-apply treatment for fungal infections of human nails.

### SUMMARY OF THE INVENTION

The current invention presents a method for treating human onychomycosis comprising contacting fungal infected human nails with copper salts, preferably copper sulfate. Copper salt compositions can be applied to human nails to prevent, detect, control and expel fungi which cause fungal infections of the nails (onychomycosis). In some treatment applications, aqueous solutions are used to expedite and simplify the application of these salts to the human nail. These aqueous solutions are generally prepared in advance of use by the patient and can be stored in a wide variety of containers to prevent evaporation prior to use.

Aqueous solutions of copper salts have a natural tendency to precipitate out of solution and form crystals or a crystalline dust at any weakness in the vapor barrier. The integrity of the container thus determines the usable shelf life of the copper salt solution. The containers are thus expensive to manufacture and subject to deterioration on the shelf. It is also an object of this invention to provide methods and devices for applying copper salts to human nails with a longer shelf life than is provided by solutions of copper salts.

In one aspect, the invention is a nail treatment composition comprising an active ingredient consisting essentially of an effective antifungal amount of a copper salt. An “effective antifungal amount” means the amount of copper salt in a composition is effective for arresting fungal growth and/or preventing infection in human nail tissue. This amount of copper salt can range from about 1% (weight/weight) to about 100% copper salt. The copper salt can be any copper salt capable of releasing copper ions in solution or when applied to a human nail in dry form. Exemplary copper salts useful in the composition of the present invention include copper acetates, copper ammonium complexes, copper carbonates, copper perchlorates, copper halides, copper gluconates, copper oxyhalides, copper lactates, copper naphthenates, copper oxides, copper quinolinates, copper selenates, copper sulfates, and copper tartrates. Preferably, the copper salt is anhydrous copper(II)sulfate or hydrated copper(II)sulfate such as copper(II)sulfate pentahydrate. Such compositions may also be used to prevent nail fungal infection. Such compositions are capable of staining fungally infected nail tissue and may therefore be used to detect fungally-infected nails. Compositions used for detection of fungal infection of nails may use copper salicylates as an active ingredient as an alternative to the foregoing copper salts.

The nail treatment composition may also comprise a topical pharmaceutical carrier, which may comprise a surfactant, a moisturizer, a preservative, a thickener, an emulsifier, an adhesive, a penetrating agent, a gel nail product formulation, a nail polish which permits continuous migration of the copper salt into the nail over time, a cuticle softening solution, or a nail conditioning formulation.

In one embodiment of the present invention, the composition comprises from about 1% (weight/weight) to about 100% copper salt, preferably copper(II)sulfate. In another embodiment, the composition comprises about 17% (weight/weight) to about 32% (weight/weight) copper salt, preferably copper(II)sulfate; preferably, about 10% (weight/weight) copper salt, preferably copper(II)sulfate. The composition may also comprise a component selected from the group consisting of a nail polish, acrylic nail product, gel nail product, cuticle softening solution, nail conditioning formulation, artificial nail product, and nail ornamental product. In another embodiment, the nail composition further comprises a fixative. In another embodiment, the nail composition further comprises an adhesive. The composition can also release the copper salt over time using a controlled-release formulation.

One embodiment of the invention uses aqueous or organic solutions of copper salts having an effective concentration for applying to nails and effecting slowing of growth or eliminating fungi on the nails. The effective concentration may be as high as 100%, i.e., solvent may be completely absent, or present in only small amounts. Solution concentrations of from about 1% to about 32% are useful in this invention, as are solution concentrations of from about 32% to about 100% (100% being dry copper salt in anhydrous or hydrated form). (Percentages are weight/weight percent.)

One embodiment of the present invention uses a copper salt in the manufacture of an antifungal nail medicament comprising an effective antifungal amount of said copper salt effective for preventing or arresting fungal invasion and/or growth in human nail tissue.

The term “copper salt” is meant to include copper cations, preferably copper (II) ions. “Migration of the copper salt” includes migration of only copper cations, and does not necessarily imply migration of the corresponding anions.

One embodiment of the present invention uses a copper salt in the manufacture of a medicament or treatment for detecting fungal infection in a human nail comprising an effective antifungal amount of said copper salt to stain fungal infected human nail tissue. The composition can comprise an active ingredient consisting essentially of a copper salt in an amount effective for persistently staining a fungal infected nail.

In another aspect, the invention is a method for treating fungal growth and/or preventing infection on a human nail comprising topically applying a composition having an active ingredient consisting essentially of an effective antifungal amount of a copper salt to the nail, wherein the effective amount arrests fungal growth and/or prevents infection. Prevention of infection in a human nail is indicated when another nail is infected, or when using artificial nails or nail ornamentals that can foster infection.

In another aspect, the invention is a method for persistent treatment of a fungal infection of a human nail comprising topically applying a composition having an active ingredient consisting essentially of an effective antifungal amount of a copper salt to the nail, wherein the effective amount capable of arresting fungal growth of nail tissue persists on the fungal infected nail tissue after repeated washing of the nail with a suitable solvent. Preferably, the method utilizes one or more of the nail compositions disclosed herein.

In another aspect, the invention is a method of detecting fungal infection in a human nail comprising topically applying a composition having an active ingredient consisting essentially of an effective antifungal amount of a copper salt to the nail, wherein the composition visibly stains any fungal infected nail tissue; washing the infected nail with a suitable solvent to remove stain from uninfected nail tissue; and equating the persistent stained nail tissue with fungal infected nail tissue.

In another aspect, the invention is a method of monitoring fungal infection in a human nail comprising, at a first time point, topically applying a composition having an active ingredient consisting essentially of an effective antifungal amount of a copper salt to the infected nail, wherein the composition visibly stains any fungal infected nail tissue; washing the nail with a suitable solvent to remove stain from uninfected nail tissue; equating the persistent stained nail tissue with fungal infected nail tissue; comparing the area of persistent stained nail tissue to the area of unstained nail tissue; at a second time point, topically applying a composition having an active ingredient comprising essentially of an effective antifungal amount of a copper salt to the infected nail, wherein the composition stains any fungal infected nail tissue; washing the nail with a suitable solvent to remove stain from uninfected nail tissue; equating the persistent stained nail tissue with fungal infected nail tissue; comparing the area of persistent stained nail tissue to the area of unstained nail tissue; wherein an increase in the area ratio of unstained nail tissue to persistent stained nail tissue at said second time point to the area ratio of unstained nail tissue to persistent stained nail tissue at said first time point indicates a decrease in fungal infection. Preferably, the method utilizes one or more of the nail compositions disclosed herein.

In one embodiment of the present invention, the copper salt composition comprises or consists essentially of one or more copper salts in a dry form, however it is understood that any person skilled in the art could choose to add an appropriate surfactant, moisturizer, preservative, thickener, emulsifier, adhesive, penetrating agent, gel nail product formulation, cuticle softening solution, nail conditioning formulation, and/or antimicrobial, if desired to modify the copper salt composition, all of which modifications are within the scope of the present invention. The copper salt composition of the present invention is also provided in time-release formulation. Methods of making such time-release formulations are known to those skilled in the art.

One embodiment of the present invention provides a method of using a copper salt composition to treat human onychomycosis by preparing a dosage delivery patch. A delivery patch is a bandage or strip, preferably an adhesive bandage or strip, and preferably a waterproof or water-resistant bandage or strip, such as a band-aid, that can be secured over a nail. The term “delivery patch” as used herein refers to a backing for a delivery device, and does not include the active ingredient or attached layers, e.g. of absorbent material. A delivery patch can be prepared, preferably during manufacture, with a deposit of an effective amount of copper salt in dry form to treat the fungal infection. The delivery patch with, as an active ingredient, an effective amount of copper salt is then secured over a nail.

In one embodiment, the delivery patch is combined with a layer of gauze or other absorbent material, which may or may not be attached to the delivery patch, and the copper salt is deposited between the absorbent material and the patch or strip. Thus, the finished product carries a powder, solid or crystalline form of the salt composition within the patch or strip, which is suitable for applying to the human nail. This type of patch or strip can be stored indefinitely using ordinary inexpensive packaging materials. Thus evaporation is not a factor in the shelf life of the product.

The dry copper salt composition can be activated by adding water or a suitable solvent to the absorbent portion of the patch or strip, so that when it is applied to the nail, the copper salt will be carried in solution to the nail to medicate the nail. The water or other solvent may also be applied after the patch or strip has been applied to the nail.

One embodiment of the invention is a method of treatment wherein a patch or strip containing a powder or solid or crystalline form of the copper salt composition is applied to the nail directly, and normal human body moisture dissolves the copper salt composition and causes the resulting solution to medicate the nail.

In one embodiment, the invention provides an antifungal copper salt composition comprised in or on a patch or strip device having a backing portion, such that an absorbent portion of the patch or strip contains a predetermined effective amount of said copper salt composition in a dry form. Such patch or strip device may contain the copper salt composition in dry form between the absorbent material and the backing. Preferably the copper salt is applied during manufacture of the device.

The patch or strip device may also contain the copper salt composition within the absorbent material as well as between the absorbent material and the delivery patch itself.

The antifungal copper salt composition may also be deposited on or added to a membrane, preferably during manufacture, such that when the device is applied to the fingernail or toenail, the composition is held in contact against the nail.

This invention also provides a thimble-shaped device comprising the dry copper salt composition designed to fit over a human digit.

This invention also provides a pliable band for application to the fingernail or toenail, the pliable band comprising the dry copper salt composition.

This invention also provides methods of treating a patient having a fungal infection of a toenail or fingernail with an amount of a copper salt composition effective to at least slow the growth of said fungal infection. Any delivery device known to the art for providing contact between a medicament and an affected area on a patient's body may be used in the treatment methods.

In one embodiment, a composition comprising a copper salt in an amount effective for arresting fungal growth and or infection, or in an amount effective to stain a fungal infected nail, allows continuous migration of the effective amount of copper salt into the nail over time. After application of the copper salt, whether in solution or dry form, the nail may be washed as part of a normal hygiene routine, or need not be washed.

Other aspects of this invention include human nails stained with a copper salt, especially portions of such human nails which are trimmings from nails stained with a copper salt.

### DETAILED DESCRIPTION OF THE INVENTION

In one aspect, the present invention is a composition for treating human onychomycosis comprising an effective antifungal amount of at least one copper salt, such as copper sulfate, which is safe for topical application to human nail tissue and an acceptable carrier. Upon application of the composition of the present invention to fungal infected human nail tissue, the fungal matter embedded in the infected nail is contacted with an effective antifungal amount of the copper salt(s). In one embodiment of the present invention, it is advantageous that the composition of the present invention be reapplied to human nail tissue after an extended period of time. In another embodiment, the composition of the present invention remains in contact with the nail for an extended period of time.

Exemplary copper salts include but are not limited to copper acetates, copper ammonium complexes, copper carbonates, copper perchlorates, copper halides, copper gluconates, copper oxyhalides, copper lactates, copper naphthenates, copper oxides, copper quinolinates copper salicylates, copper selenates, copper sulfates, and copper tartrates can be used. Preferably, the copper salt is copper sulfate, also known in the art as copper sulfate, cupric sulfate, copper(II)sulfate, Roman vitriol, Fehling solution A, salzburg vitriol, sulfuric acid copper(2+) salt, blue copper, copper monosulfate, CUSO4, copper(2+)sulfate, cupric sulfate anhydrous, incracide E 51, blue stone, hi-chel, blue vitriol, natural chalcanthite, and other names. The most preferred form of copper sulfate utilized in the present invention is copper(II)sulfate pentahydrate. The copper salts useful in the present invention are commercially available from general chemical suppliers such as Sigma Chemical Company (St. Louis, Mo.), Aldrich (Milwaukee, Wis.), and Mallinckrodt Laboratory Chemicals (Phillipsburg, N.J.). It is understood that the present invention can utilize copper salts having a wide range of purity, including but not limited to industrial, technical, or reagent grade, provided that any impurity present in the copper salt is not harmful to humans on topical application.

In one embodiment, the composition of present invention preferably comprises an amount of one or more copper salts equivalent to the amount of copper found in a 1-32% (weight/weight) aqueous composition of copper (II) sulfate at room temperature. However, practical concentrations can vary from trace amounts to a paste of approximately 100% (weight/weight) of copper salts such as copper sulfate, depending upon the frequency of application, type of application, type of carrier used, and toxicity and/or patient sensitivity to the copper salt as disclosed herein. The preferred concentration of copper salts useful in the present invention can vary depending upon the type of application, wherein the concentration of copper salt in a composition for the treatment of fungal infection is preferably higher than the concentration of copper salt used in nail polish or adhesive. Moreover, the preferred concentration of copper salt useful in the present invention for the treatment of fungal infection can vary depending upon the preferred treatment regimen. For example, a stronger concentration of copper salt is preferable in a composition which is infrequently applied; however, for a patient having sensitivity to copper salt, a composition with a lower concentration of copper salt applied more frequently would be preferred. For the treatment of fungal infection, a preferred composition of the present invention comprises copper (II)sulfate at a final concentration of about 1%-32% (weight/weight) anhydrous copper(II)sulfate or copper(II)sulfate pentahydrate; preferably, 7%-20% (weight/weight); most preferably, 10% (weight/weight) at room temperature.

An acceptable topical pharmaceutical carrier useful in the composition of the present invention is any solvent system or carrier which stably solubilizes or suspends the copper salt and is tolerated by human tissue. One type of acceptable carrier can solubilize the copper salt(s) and prevent subsequent recrystallization of the copper salt(s) over time. Any carrier suitable for the active ingredient of the current invention is herein considered part of this application. The cosmetic arts are rich in chemicals suitable for applying to human skin and nails. The following compounds are merely examples and not meant to be limiting in any way. Carriers that may be used in conjunction with copper salts include moisturizers such as dimethicone silicone, isopropyllanolate, lanolin, oleic acid, panthenal, and stearic acid. Surfactants or detergents such as sodium laureth sulfate can be added to the copper salt composition of the present invention to provide foaming, cleansing, wetting, emulsifying, solubilizing, and dispersing properties to the solution. Preservatives that may be used in a copper salt composition may include tocopherol and EDTA. Additionally, antimicrobials such as parabens, quaternium-15, methylisothiazolinone, DMDM hydantoin, and phenoxyethanol may also be used in a copper salt formulation. It may be advantageous to include thickeners like microcrystalline waxes, polyethylene thickeners, and carbomer thickeners, in a copper salt composition. Solvents that may be included in a copper salt formulation include water, a combination of water and glycerol, and a combination of water and a volatile organic solvent(s), including but not limited to butylene and propylene glycol, cyclomethicone, and glycerin. Emulsifiers such as glyceryl monostearate, lauramide DEA, and polysorbates, may be advantageous to add to a copper salt composition. To increase the penetration of the copper salt composition into the fungal infected nail, penetrating agents can optionally be added. It is to be well understood that any person skilled in the art could choose appropriate surfactant, penetrating, moisturizing, antimicrobial, and emulsifying agents if desired to modify the copper salt composition, all of which are contemplated by the present invention.

Any carrier or delivery device known to the art may be used for contacting the infected nail with the copper salts of this invention, either in dry form or in solution or suspension. No matter what type of carrier or delivery device is used, the present invention provides the advantage of not requiring daily application of the medicament. Applications of the copper salts may be done weekly or monthly. However, applications should in any event be done more often than at about two-month intervals in order to prevent resurgence of fungi.

For treatment of fungal infection, an effective antifungal amount of copper salt(s) can also be added to existing nail products such as a commercially available nail polish which allows migration of the copper salt(s) into the nail, gel nail products, cuticle softening solutions, nail conditioning preparations, or various products used in association with artificial nails or ornamentals. By adding a fixative, the copper salt composition can be applied as a film to the nails prior to the application of nail polish, artificial nails or ornamentals. Applying the composition of the present invention in a nail polish which allows penetration of copper salt into the nail prior to the application of nail polish, artificial nails or ornamentals provides the advantage of having the copper salt-containing product remaining in contact with the nail for an extended period of time. Also, the nail polish can optionally comprise pigment when it is advantageous to minimize the appearance of staining of nail tissue caused by the copper salt. It is contemplated that one product or a combination of products comprising copper salt in an effective antifungal amount can be used.

One embodiment of the present invention is a method for treating onychomycosis in humans comprising applying a copper salt composition, preferably with an absorbent swab, felt, gauze, eyedropper or brush, to the fungal infected nail. Preferably, the copper salt composition is applied for approximately 30 minutes and allowed to air dry. On drying, a powder coating of copper salt covers the treated skin and nail. On washing with water or other suitable solvent, the porous (infected) portion of the nail may be permanently stained from the copper salt, while normal nail tissue and skin is temporarily stained and returns to normal coloration. A suitable solvent for washing is any solvent which is capable of solubilizing the copper salt without causing damage to the nail tissue or surrounding skin. The washing solvent and the carrier in the copper salt composition can be the same or different for copper (II) sulfate, water is a preferred solvent for washing.

Repeated washing of hands or feet over time generally results in further migration of the copper salt into the infected nail. Thus, normal washing does not interrupt the treatment process or require repeated application of the copper salt solution after each washing. As the treated nail grows, a narrow band having a reddish pink color may appear immediately behind the blue-stained infected nail portion. As new, healthy nail tissue appears, the stained infected portion of the nail grows beyond the nail bed and is trimmed away. Application of the copper salt composition is repeated upon fading of the stain, more preferably at least once monthly until signs of infection are eliminated.

In addition to the copper salt treatment of the present invention, the method to treat onychomycosis in humans can include processes that are generally part of nail health maintenance including trimming and sanding the nail, keeping the nail dry, and treating the affected areas with chemicals to enhance the absorption of and/or prevent the leaching out of the copper salt from the nail over time. For example, clear or colored nail polish can be applied to the copper salt treated nail. Likewise, stained, treated nails can be cosmetically disguised by the application of colored nail polish.

Another aspect to the method of using a copper salt composition to treat human onychomycosis includes treating nails before they present the symptoms of onychomycosis. For example, if one toe nail exhibits the symptoms of onychomycosis, all toe nails can be treated even if symptoms are not yet apparent. Furthermore, since the copper salt composition will stain only the nail portions exhibiting fungal invasion, the progress of the fungal infection within one nail can be monitored, or possible spreading to other nails can be detected. For example, a staining copper salt, preferably copper sulfate, can be added to routine soaking solutions used in the art prior to manicuring or pedicuring.

The copper salt composition of the present invention can also be used to prevent onychomycosis as well as the growth of noninvasive fungi residing on the surface of human nails, for example, fungi commonly seen in association with the use of artificial nails or ornamentals. In one aspect, a copper salt can be added to nail polish formulations which allow penetration of copper salt into the nail, acrylic nail products, gel nail products, cuticle softening solutions, nail conditioning preparations, or various products used in association with artificial nails or ornamentals to effectively prevent the growth of fungi residing on the nail. Preferably, the copper salt can be applied in a time-release formulation. One product or a combination of products comprising copper salt can be used in the prevention of onychomycosis or superficial fungal growth.

Because of the potential staining properties of the copper salt composition of the present invention, containment of the copper salt composition during shipping and storage as well as upon application to infected nails is preferable. One preferred method is a waterproof paper, plastic or foil package containing an absorbent swab or felt or gauze pad saturated with copper salt composition, whereupon tearing away a portion of the package exposes the copper salt composition saturated absorbent tip of the swab or a portion of the copper salt composition saturated felt or gauze pad which can be directly applied to the infected nail. In another preferred method, the copper salt composition is encapsulated in a breakable ampule, the breakable ampule is enclosed in a pliable container capable of confining the ampule upon breakage and having one sealed end and one opened end that is packed with an absorbent material, and the whole device is enclosed in a waterproof paper, plastic or foil package. On use, the ampule is broken to release the copper salt composition, the absorbent material is saturated with the copper salt composition, and a portion of the package is torn away to expose the copper salt composition saturated absorbent material which can be directly applied to the infected nail.

An acceptable method to minimize the expense of packaging and maximize the useful shelf life of a copper salt composition for application to the human nail is to store the copper salt in a solid, dry, crystalline form ready for use.

The present invention further provides devices and methods in which the copper salt composition in dry form is placed on the patch or strip in such a way that body moisture releases the copper salt composition into solution for contact with the nail over time, reducing the need for frequent changing of the patch or strip. For example, in one embodiment of this invention, a portion of the dry copper salt composition is integrated into a layer of gauze or other absorbent material attached to the patch or strip, and a further portion of the dry copper salt composition is placed between the absorbent material and the remainder of the patch or strip. This placement of the latter portion of the composition further from the nail delays entry of this portion of the composition into solution and delays its contact with the nail, thereby providing a fresh application of the copper salt composition after the copper salt composition on the absorbent material has been exhausted.

Such a patch or strip, e.g. a band-aid, is a convenient medical tool with which to apply the copper salt to the human nail. However, any absorbent material or gauze in contact with the nail may cause even distribution of moisture throughout the absorbent material itself, resulting in much of the solution of the copper salt composition at least initially being retained in the gauze itself rather than being applied to the crevice between the nail and cuticle. When infection is severe and direct application of the copper salt to the nail is of primary importance, a patch or strip without an absorbent material can be more immediately effective.

Another aspect of the present invention provides that a dry copper salt composition is deposited on a membrane of the type used with a band-aid style backing without an absorbent material, so that when said membrane is applied to the nail, the dry salt composition on the membrane is activated by adding water or a suitable solvent to dissolve the copper salt and cause the resulting solution to medicate the nail. In this embodiment, the membrane bearing the copper salt composition can also be applied to the nail directly and normal human moisture allowed to dissolve the copper salt composition and cause the resulting solution to medicate the nail.

The permeability of the backing and the membrane are controlled during manufacture to limit but not prevent the transfer of moisture to and from the nail.

Another aspect of the invention provides a method of applying the copper salt composition to the nail in a manner which holds the composition in place without the use of patches, strips, band-aids, membranes or thimble-like devices. This aspect of the invention provides a pliable compression band which is slipped over the fingernail or toenail. The pliable band grips the finger or toe with an elastic compression. The pliable band exposes a useful amount of the tip of the finger or toe for normal activities and makes the treatment more acceptable to the subject. The pliable band is also manufactured to contain the deposit of copper salt composition in a manner similar to that described for patches, strips, band-aids and membranes.

The permeability of both the pliable thimble-like device and the pliable band is controlled during manufacture to limit the transfer of moisture to and from the nail.

Another aspect of this invention is the use of aqueous or organic solutions of copper salts or suspensions or saturated solutions comprising undissolved copper salts having an effective amount of copper salts for applying to nails to effect slowing of growth or elimination of fungi on the nails and surrounding tissues. The copper salts may be present in the aqueous or organic carrier in trace amounts, e.g. about 0.01%. Preferably the copper salts are present in the carrier in amounts of at least about 0.0055%. The copper salts may also be present in the carrier in amounts of at least about 55%. The percent copper salts in the carrier may be as high as 100%, i.e., carrier may be completely absent, or may be present in only small amounts. Copper salts may be present in the carrier in amounts from 1%, 5%, or 10% up to the solubility limit of the copper salts in the carrier and beyond. For example, the solubility of copper sulfate in water is 32%. Additional copper salts may also be present in the carrier in suspension or as a sediment. (Percentages are weight/weight percent.)

### EXAMPLE 1

### Toe Nail Treatment

A male patient diagnosed as having a fungal infected toe nail, began treatment by applying an aqueous 10% (w/v) copper(II)sulfate pentahydrate solution to the infected nail using a copper sulfate saturated gauze pad. The copper sulfate solution was applied to the infected toe nail for approximately thirty minutes and then allowed to air dry. Excess copper sulfate powder was removed by washing two times with water. The infected portion of the patient's nail had a bluish color. As the treated nail grew, healthy nail tissue appeared at the base of the nail bed while the infected portion of the nail was pushed off the nail bed and periodically trimmed away. Over time, the fungal infection did not spread to healthy nail tissue and the stained, treated portion of the nail was removed.

### EXAMPLE 2

### Toe Nail Treatment

A male patient diagnosed as having a fungal infected toe nail began treatment by applying with a cotton swab an aqueous 10% (w/v) copper(II)sulfate pentahydrate solution to the infected nail as well as to all nails on the same foot. The copper sulfate solution was applied to his toe nails for approximately thirty minutes and then allowed to air dry. Excess copper sulfate powder was removed by washing two-three times with water. The patient's infected nail had a bluish color where the infection was present. All other nails return to normal coloration, indicating that the fungal infection had not spread from the infected toe to neighboring toe nails on the same foot. As the treated nail grew, healthy nail tissue appeared at the base of the nail bed while the infected portion of the nail was pushed off the nail bed and periodically trimmed away. Monthly, the copper sulfate solution was applied as given above to the infected nail and to all nails on the same foot. The infected portion of the patient's nail continued to have a bluish color. All other nails returned to normal coloration on washing, indicating that the fungal infection still had not spread from the infected toe to neighboring toe nails on the same foot. Over time, the fungal infection did not spread to healthy nail tissue and the stained, treated portion of the nail was removed, resulting in a normal healthy nail.

### EXAMPLE 3

### Fingernail Treatment

A female patient diagnosed as having a fungal infected fingernail began treatment by applying with a cotton swab an aqueous 10% (w/v) copper(II)sulfate pentahydrate solution to the infected nail as well as to all nails on the same hand. The copper sulfate solution was applied to her fingernails for approximately thirty minutes and then allowed to air dry. Excess copper sulfate powder was removed by washing two-three times with water. The patient's infected nail had a bluish color where the infection was present. All other nails returned to normal coloration, indicating that the fungal infection had not spread from the infected fingernail to neighboring fingernails on the same hand. The patient routinely covered her fingernails in an opaque, colored nail enamel which completely disguised the infected nail. As the treated nail grew, healthy nail tissue appeared at the base of the nail bed while the infected portion of the nail was pushed off the nail bed and periodically trimmed away. Monthly, the copper sulfate solution was applied as given above to the infected fingernail and to all fingernails on the same hand. The infected portion of the patient's nail continued to have a bluish color. All other nails returned to normal coloration on washing, indicating that the fungal infection still had not spread from the infected fingernail to neighboring fingernails on the same hand. Over time, the fungal infection did not spread to healthy nail tissue and the stained, treated portion of the nail was removed, resulting in a normal healthy nail.

### EXAMPLE 4

### Fingernail Treatment

A female, 30 years old, was diagnosed as having an infection of onychomycosis of the right index fingernail. The nail was separating from the nail bed along the right side of the nail and along the cuticle, leaving the nail hinged along the left side of the nail only. The patient required a bandage to prevent the nail from tearing free. The right side of the nail displayed the characteristic yellowing and rough, thick texture associated with the fungal infection. Prior to the first treatment on Day 1, all fingernail polish was removed from the nail using over-the-counter nail polish remover. A bandage was soaked with a preparation consisting of a 10% aqueous copper sulfate solution with a trace of detergent added to act as a surfactant. The bandage was applied to the infected nail and secured in place. Care was given to ensure that the solution soaked into the crevice along the edge of the nail bed and cuticle. Subsequent hand washing provided additional wetting which drove more of the copper sulfate solution into the nail bed and surrounding crevice. The bandage was worn for two days and then replaced with a clean dry bandage to protect the nail from snagging. The treatment was repeated on Day 31 and again on Day 45; no further treatments were given. Between treatments, the patient was permitted to resume the use of fingernail polish between treatments. On Day 132, the nail was examined. The patient was no longer wearing a dry bandage to protect the nail. New pink nail growth was present from the cuticle to a point midway along the nail. The new portion of the nail was attached normally on both sides of the nail from the cuticle to the midpoint. There was a slight “keloid type” ridge across the nail at the midpoint. The old fungal infected nail was still separated from the midpoint of the right side of the nail to the end of the nail. The new growth immediately behind the “keloid type” ridge was rough in texture, typical of nail regrowth following a fungal infection. However, the nail newly emerging from the cuticle was smoother, like an uninfected nail.

It is to be understood that the above description is of preferred exemplary embodiments of the invention and is intended to be illustrative of the invention, but is not to be construed to limit the scope of the invention in any way. Modifications may be made in the composition features of the invention without departing from the scope of the invention. It will be readily apparent to those skilled in the art that alternative materials may also be utilized without departing from the scope of the invention.

What is claimed is:

1. A human nail treatment composition comprising:
a. an active ingredient consisting essentially of a copper salt in an amount effective for arresting fungal growth in human nail tissue; and
b. a topical pharmaceutical carrier;
wherein said composition is capable of persistently staining a fungally-infected nail.

2. The composition of claim 1 which allows continuous migration of an effective antifungal amount of said copper salt into human nail tissue over time.

3. The composition of claim 1 wherein said carrier comprises a component selected from the group consisting of a surfactant, a moisturizer, a preservative, a thickener, an emulsifier, an adhesive, a penetrating agent, a gel nail product formulation, a cuticle softening solution, and a nail conditioning formulation.

4. The composition of claim 1 wherein said copper salt is anhydrous or hydrated copper(II)sulfate.

5. The composition of claim 4 wherein said composition comprises at least about 1% (weight/weight) anhydrous or hydrated copper(II)sulfate.

6. The composition of claim 4, wherein said composition comprises 10% (weight/weight) anhydrous or hydrated copper(II)sulfate.

7. The composition of claim 1 comprising a controlled-release formulation wherein said copper salt is released over time.

8. The composition of claim 1 wherein said copper salt is selected from the group consisting of copper acetates, copper ammonium complexes, copper carbonates, copper perchlorates, copper halides, copper gluconates, copper oxyhalides, copper lactates, copper naphthenates, copper oxides, copper quinolinates, copper selenates, copper sulfates, and copper tartrates.

9. A product for treating a fungal infection of a human nail comprising the composition of claim 1 and also comprising a waterproof package containing an absorbent swab or pad saturated with an effective amount of said copper salt.

10. A product for treating a fungal infection of a human nail comprising the composition of claim 1 also comprising a breakable ampule enclosed in a pliable container capable of confining the ampule upon breakage, said container having one sealed end and one opened end packed with an absorbent material, said ampule and container being enclosed within a waterproof package.

11. A human nail treatment composition comprising:
a. a copper salt in an amount effective for arresting fungal growth in human nail tissue; and
b. a component selected from the group consisting of a surfactant, a moisturizer, a preservative, a thickener, an emulsifier, an adhesive, a penetrating agent, a gel nail product formulation, a cuticle softening solution, and a nail conditioning formulation.

12. A method for treating, preventing, or detecting a fungal infection of a human nail comprising applying to said nail a composition having an active ingredient consisting essentially of an antifungal amount of a copper salt effective for arresting fungal growth in human nail tissue.

13. The method of claim 12 which is a method for treating a fungal infection of a human nail.

14. The method of claim 12 which is a method for preventing fungal infection of a human nail.

15. The method of claim 12 which is a method for detecting fungal infection of a human nail, wherein said nail is persistently stained by said copper salt.

16. A method of claim 12 wherein said composition also comprises a component selected from the group consisting of a surfactant, a moisturizer, a preservative, a thickener, an emulsifier, an adhesive, a penetrating agent, a gel nail product formulation, a nail polish which permits migration of the copper salt into the nail, a cuticle softening solution, and a nail conditioning formulation.

17. The method of claim 12 wherein said copper salt is copper(II)sulfate anhydrous or hydrated copper(II)sulfate present in an amount of at least about 1%.

18. A method for treating, preventing, or detecting a fungal infection of a human nail comprising applying to said nail a composition comprising an antifungal amount of a copper salt effective for arresting fungal growth in human nail tissue, wherein said composition allows continuous migration of an effective antifungal amount of said copper salt into said nail over time.

19. The method of claim 18 which is a method for treating a fungal infection of a human nail.

20. The method of claim 18 which is a method for preventing a fungal infection of a human nail.

21. The method of claim 18 which is a method for detecting a fungal infection of a human nail, wherein said copper salt persistently stains infected nail.

22. A method for treating, preventing, or detecting a fungal infection of a human nail comprising applying to said nail a composition comprising a copper salt in an amount effective for arresting fungal growth in human nail tissue, said copper salt being selected from the group consisting of copper acetates, copper ammonium complexes, copper carbonates, copper perchlorates, copper halides, copper gluconates, copper oxyhalides, copper lactates, copper naphthenates, copper oxides, copper quinolinates, copper selenates, and copper sulfates including copper(II)sulfate, copper tartrates, and when the method is for detecting a fungal infection, copper salicylates.

23. The method of claim 22 which is a method for treating a fungal infection of a human nail.

24. The method of claim 22 which is a method for preventing a fungal infection of a human nail.

25. The method of claim 22 which is a method for detecting a fungal infection of a human nail, wherein said copper salt persistently stains infected nail.

26. A human nail stained with an effective antifungal amount of a copper salt for arresting fungal growth.

27. A portion of a human nail of claim 26 stained with a copper salt which has been trimmed from said human nail.

28. A nail treatment product comprising a delivery patch capable of being applied to a human nail, said product comprising as an active ingredient an effective antifungal amount of a copper salt in dry form.

29. The product of claim 28 further comprising a layer of absorbent material.

30. The product of claim 29 wherein said copper salt is deposited between said layer of absorbent material and said delivery patch.

31. The product of claim 29 wherein said copper salt is deposited within said layer of absorbent material.

32. A method of treating fungal infection of a human nail comprising applying to said nail a product of claim 28.

33. The method of claim 32 wherein said product has a layer of absorbent material.

34. The method of claim 33 further comprising adding water or a suitable solvent to the absorbent material.
